(19) United States
(12) Patent Application Publication
(10) Pub. No.: US 2010/0172993 A1
(43) Pub. Date: Jul. 8, 2010

(54) PARTICLES FOR DELIVERY OF ACTIVE INGREDIENTS, PROCESS OF MAKING AND COMPOSITIONS THEREOF

(76) Inventors: Amarjit Singh, Mumbai (IN); Sarabjit Singh, Mumbai (IN); Paramjit Singh, Mumbai (IN); Rajesh Jain, New Delhi (IN)

Correspondence Address: LADAS & PARRY LLP, 26 WEST 61ST STREET, NEW YORK, NY, 10023, US

(21) Appl. No.: 12/377,185

(22) PCT Filed: Aug. 10, 2007
(86) PCT No.: PCT/IN07/00340

§ 371 Date: Feb. 11, 2009

(30) Foreign Application Priority Data
Aug. 11, 2006 (IN) 1276/MUM/2006
Apr. 3, 2007 (IN) 666/MUM/2007

(51) Int. Cl. A61K 9/14; A61K 31/568; A61K 31/565; A61K 38/02; A61K 39/00; A61K 39/395; A61K 8/23; A61K 8/19; A61K 31/715; A61K 8/60; A61K 8/73; A61K 8/64; A61K 8/66; A61K 8/98; A61K 8/97; A61K 31/137; A61K 31/522; A01N 37/18; C07C 211/30; C07H 15/16; A61K 31/7056; A61K 38/22; C07D 473/18; C07C 233/65; C07K 14/575; A61K 31/52; A61K 38/12; A61K 33/18; A61K 31/635; A61K 36/61; A61K 36/53; A61K 36/534; A61K 36/54; A61P 31/10; A61P 31/12; A61P 33/10; A61P 17/06; A61P 17/10; A61P 11/06; A61P 19/02; A61P 25/24; A61P 3/10; A61Q 5/12; A61Q 5/10; A61Q 5/08; A61Q 17/04; A61Q 15/00; A61Q 13/00

(52) U.S. Cl. 424/489; 514/178; 514/182; 514/2; 424/184.1; 424/130.1; 424/702; 424/59; 514/54; 514/23; 424/94.1; 424/520; 424/725; 424/65; 424/70.1; 424/62; 514/655; 514/263.38; 514/617; 564/387; 536/16.5; 514/24; 514/12; 544/276; 564/182; 530/399; 514/263.4; 514/11; 424/670; 514/157; 424/133.1; 424/154.1; 424/742; 424/745; 424/747; 424/739; 8/405; 512/2; 977/775

(57) ABSTRACT

The present invention discloses compositions having particles comprising, inorganic element; one or more active ingredient and optionally a release rate modulating agent, suitable for the delivery of active ingredients to human and animal tissues. The particles are nanoparticles or microparticles or mixtures thereof, made preferably by sol-gel method. The compositions are useful for application to the topical or mucosal surfaces preferably in the form of creams, gels, lotions, dry powders, spray, foam and other suitable forms.

### FIELD OF THE INVENTION

The present invention is directed to the field of delivery of active ingredients. It relates to particles for the delivery of active ingredient(s) in mammalian systems, process of making them and their compositions. More specifically, the present invention relates to microparticles and nanoparticles for the delivery of active ingredients to topical and mucosal surface.

### BACKGROUND OF THE INVENTION

Modern drug delivery technologies have led to sophisticated systems which allow targeting and controlled release of active ingredients in mammalian systems. Delivery systems which are in nano-scale dimensions provide an efficient, less risky solution to many drug delivery challenges. They can be used for targeting to highly specific sites of action, and due to their small dimensions, can be used for delivery to tissues which are inaccessible to the more conventional delivery agents. Polymer based nanoparticles are known for such systems. However, they utilize costly raw materials, are often expensive to manufacture and are not very scale-up friendly.

In recent years, metal oxide based systems have been developed by sol gel technique. This technique refers to a low-temperature method using chemical precursors which can produce diverse types of ceramics and glasses. It enables researchers to design and fabricate a wide variety of different materials with unique chemical and physical properties. The sot-gel materials are based on silica, alumina, titanium and other compounds. The technology allows fabricating: monolithic and porous glasses, fibers, powders, thin films, nanocrystallites, photonic crystals etc.

Recently, biological applications, where biomolecules (such as proteins, enzymes, antibodies etc.) are incorporated into the sol-gel matrix, have been studied. Applications include biosensors in diagnostic applications, environmental testing, biochemical process monitoring and food processing. In 1983, Unger and coworkers used sol-gel derived silica gel for drug delivery applications (Unger, et. al 1983, “The use of porous and surface modified silica as drug delivery and stabilizing agents” Drug Dev. Ind. Pharm. 9, 69-91). Since then, the use of silica based materials, especially the silica based xerogels and mesoporous structures as carrier systems for controlled delivery of drugs, has been explored.

Application of inorganic element based systems for delivery of active ingredients is one of the current research interests, and there is a need to develop novel technologies in this field which can be used for the controlled delivery of active ingredients to human and animal tissues. Especially, there is a need for carrier systems which are easier to produce, biocompatible and easily and predictably biodegraded, and retained at the site of action when applied to topical or mucosal surface.

Metal elements such as titanium, magnesium, calcium, aluminum, silver, zinc and others are present in human bodies some of them are present at least in trace amounts and have been used in various biocompatible products. They are also easily available.

U.S. Pat. No. 6,710,091 discloses a process for preparing nanoparticulate redispersible Zinc oxide gels. The process leads to Zinc oxide particles having an average primary particle diameter of less than 15 nm. The application discloses use of Zinc oxide particles as UV absorbers, in plastics, paints, coatings and for the protection of UV-sensitive organic pigments. It does not disclose Zinc oxide structures for encapsulation of any species, nor does it disclose the application of Zinc oxide particles in drug delivery.

United States Application No. 2005/0226805 describes a process for producing micro-mesoporous metal oxide having average pore size of not more than 2 nm and not less than 1 nm, by sol-gel synthesis, using non-ionic surfactant as a template. The mesoporous metal oxides of the invention are expected to be useful in catalysts, sensors or semiconductors. The application does not disclose any use in drug delivery or encapsulation of any active species.

United States Application No. 2005/0003014 describes synthetic inorganic nanoparticles as carriers for ophthalmic and otic drugs. The carriers of the invention are mainly water swellable clays, although other materials, such as zeolites, silica, aluminum oxide, titanium oxide, cerium oxide and zinc oxide, are also included. The materials are finely dispersed in a vehicle to form clear low viscosity gels. The nanoparticles of the invention function as chemically inert carriers for the drugs, possibly only by association. Compositions of active ingredients in nanostructures for their controlled release are not disclosed. Also, the nanoparticles may not be biodegradable.

United States Application No. 2006/0171990 describes drug delivery materials which include active compounds encapsulated within a polymeric shell; the encapsulated compounds are then incorporated in a matrix prepared by sol-gel technology. The matrices thus prepared are used for porous or non-porous film coatings for implants such as stents, bone grafts, prostheses etc. The invention thus discloses a two step process, where first the active compound is encapsulated into a conventional polymeric shell, before dispersing the encapsulated particles into a matrix prepared by sol-gel process. The application does not disclose biocompatible inorganic nanostructures which deliver the active ingredients and modulate their release.

United States Application No. 2006/0194910 invention illustrates a stabilizer for polymers and a stabilized polymer composite. The stabilizer for polymers is in the form of a ZnO nanoparticle that, when combined with a desired monomer, polymer or copolymer provides a stabilized polymer composite with superior thermal stability. However, the invention does not disclose any application in the field of drug delivery.

U.S. Pat. No. 4,895,727 discloses a method for inducing a reservoir effect in skin and mucous membranes so as to enhance penetration and retention of topically applied pharmacologically active therapeutic and cosmetic agents therein. The invention also relates to topical treatment methods involving such reservoir effect enhancers, and to pharmaceutical compositions containing them. The additives of this invention are water-soluble zinc-containing compounds, preferably zinc halide, zinc sulfate, zinc nitrate, zinc acetate, and/or zinc stearate, and most preferably zinc chloride. Wherein, these water soluble zinc-containing compounds act as potentiators, for pharmacologically active agents.

United States Application No. 2005/0260122 is directed to sol-gel methods in which metal oxide precursor and an alcohol-based solution are mixed to form a reaction mixture that is then allowed to react to produce nanosized metal oxide particles. The present invention can provide for nanosized metal oxide particles more efficiently than the previously-described sol-gel methods by permitting higher concentrations of metal oxide precursor to be employed in the reaction mixture. However, the invention does not disclose any application in the field of drug delivery.

U.S. Pat. No. 5,989,535 describes a composition which includes a bioadhesive/mucoadhesive polymer in an emulsion or suspension form along with a treating agent. The treating agent could be as simple as water as in the case of mucoadhesive moisturizing agent. The bioadhesive/mucoadhesive polymer is a water dispersible high molecular weight crosslinked polyacrylic acid copolymer with free carboxylic acid groups further crosslinked with a combination of mono, di and polyvalent metallic cations or anions to obtain, crossed linked co-polymers of high molecular weight with reduced viscosity, solubility and having enhanced bioadhesive properties. Such compositions can be used to administer drugs systematically or locally in sustained or immediate release dosage forms, wherein the compositions can be formulated as creams, gels, suspensions, capsules and others.

U.S. Pat. No. 6,998,137 is related to compositions for the modulated release of one or more proteins or peptides in a biological environment. Such compositions comprise of (i) sparingly soluble biocompatible particle selected from zinc salts, zinc oxides, magnesium salts, magnesium oxides, calcium salts and calcium oxides, (ii) protein or peptide deposited onto the particle and (iii) a polymer matrix. The protein or peptide deposited onto the particle and polymeric matrix by, adsorption, absorption or co-precipitation. The patent does not describe inorganic nanostructures containing release rate modulating agent, prepared by sol-gel method for delivery of the active ingredients to topical and mucosal tissues.

PCT Publication No. WO2006/061835 describes nanoparticles-entrapping spherical composites, composed of a metal oxide or semi-metal oxide and a hydrophobic polymer. The spherical composites are characterized by well-defined spherical shape, a narrow size distribution and high compatibility with various types of nanoparticles. Further disclosed are processes for preparing the nanoparticles-entrapping spherical composites and uses thereof. Biocompatible organic-inorganic particles comprising active for controlled delivery are beyond the scope of this invention.

United States Application No. 2004/0109902 claims an aqueous preparation for topical application comprising equimolor amounts of a zinc salt and clindamycin phosphate for use in the treatment of dermatoses. The formulations are useful especially for the treatment of acne or rosacea, and are such that they have a very low systemic levels of clindamycin. Compositions comprising active ingredients, inorganic elements and optionally release rate modulating agents for delivery to animal and human tissues are not disclosed.

Present invention addresses the need in the art for a composition of active molecules, especially for better local delivery of active ingredients on the surface of skin or mucosal surfaces. This technology for the delivery of active ingredients offers advantages like ease of use, better retention at site of action, effective rates of absorption, controlled release over a desired period of time, dose reduction and better cosmetic and aesthetic compliance. Besides that the compositions of the present invention are not irritating to skin and not visible when applied to skin or mucosal surfaces, they are also easy to apply, and have a better patient compliance.

### BRIEF DESCRIPTION OF THE INVENTION

The invention is directed to particles, having active ingredient(s) especially but not limited to pharmaceutical and cosmetic ingredient(s) along with inorganic element(s) and optionally, having release rate modulating agent(s). It is further directed to process of making these particles and compositions for delivery of active ingredients to human and animal tissues. The said particles are either nanoparticles or microparticles or mixtures thereof. The present invention particularly is directed to the use of nanoparticles or microparticles comprising inorganic materials; active ingredient(s), and optionally release rate modulating agents for topical and mucosal applications. The particles function as a carrier or depot for one or more active ingredients and other components of the compositions.

The present invention is believed to have advantages over the existing technologies for delivery of active ingredients to topical and mucosal surfaces. For example, the compositions developed are particularly well suited for controlled delivery of the active agent(s). The particles of the present invention offer advantages over current state of the art for delivery of agent(s) such as having higher surface area hence better applicability and retention at the site of action leading to reduced frequency of application and ability to form translucent to clear gel or non-gritty powder when dispersed. Such preparations are non-irritating to the topical or mucosal surface and offer an added advantage of being non visible immediately upon application.

Compositions of such particles have better consumer acceptance due to the superior physical characteristics compared to the marketed products. These types of compositions can be used for local application of drugs in controlled release dose profiles.

In preferred embodiments composition of the invention is formulated as cream, lotion, gel, paste, powder, spray, foam, roll-on, deodorant, oil, patch, suspension, ointment, or an aerosol, useful for topical application to skin and mucosal surfaces.

In one of the preferred embodiments composition of the invention is formulated as dry powder for topical and mucosal application.

Various methods are known for the preparation of inorganic particles but do not generally include active molecules or other agents along with the inorganic element. The present invention is directed to the process of making said inorganic particles especially by novel sol-gel methods, in which an inorganic precursor, an alkali and solvent is mixed along with active molecule and optionally other agents to form a reaction mixture which when allowed reacting produces micro or nanosized inorganic particles. The method of the present invention is inexpensive and easy for preparing nanosized inorganic particles as compared to previously-described sol-gel methods.

The present invention is directed to producing compositions of particles comprising inorganic element, active ingredient(s) and optionally release rate modulating agent(s) having average particle diameter of less than about 100 μm. In preferred embodiment particles are nanoparticles having average particle diameter of less than about 2000 nm. The average particle diameter of the nanoparticles can be modulated by adjusting reaction parameters, particularly temperature, duration of reaction and, the ratio of inorganic precursor to the basic species within the reaction mixture.

The compositions of the present invention provide controlled release of active ingredient(s); have better retention at the site of action, reduced frequency of administration and better patient compliance.

In an embodiment of the invention, the invention also relates to a kit comprising a delivery device; the composition having particles comprising: inorganic element(s), one or more active ingredient(s), optionally release rate modulating agent(s) and instructions for its use; for the delivery of the composition to topical or mucosal surfaces. The delivery device comprises a pressurized or non-pressurized dispensing device or applicator or mechanical device, which delivers the composition to the topical or mucosal surfaces. In a preferred embodiment the delivery device is capable of delivering metered dose of the composition to the topical or mucosal surfaces.

The invention is further directed towards a method for treating a mammal, including a human, with the compositions of the invention.

It is to be understood that both the foregoing general description and the following brief description of the figure and detailed description are exemplary and explanatory and are intended to provide further explanation of the invention as claimed. Other objects, advantages, and novel features will be readily apparent to those skilled in the art from the following detailed description of the invention.

### DESCRIPTION OF THE DRAWINGS

FIG. 1: shows a specially designed drug release assembly for testing in-vitro release of nanoparticulate or microparticulate composition. The assembly has the following components temperature sensor probe (1), sampling probe (2), paddle (3), powder in dialysis sack with metal disk tide with rubber band (4), dialysis bag with air sac and powder dispersion (5).

FIG. 2: shows results of a drug release study (acyclovir) in pH 4.5 acetate buffer/500 ml using USP paddles floating dialysis bag/50 rpm (n=3).

### DETAILED DESCRIPTION OF THE INVENTION

Before the present invention is disclosed and described, it is to be understood that this invention is not limited to the particular process steps and materials disclosed herein, as such process steps and materials may vary to some degree. It is also to be understood that the terminology used herein is used for the purpose of describing particular embodiments only and is not intended to be limiting as the scope of the present invention will be limited only by appended claims and equivalents thereof.

It must be noted that, as used in this specification and the appended claims, singular forms of “a,” “an,” and “the” include plural referents unless the content clearly dictates otherwise.

“Biocompatible” shall mean any substance that is not toxic to the body or biological environment. A polymer or polymeric matrix is biocompatible if the polymer, and any degradation products of the polymer, are non-toxic to the recipient or biological environment and also present no significant deleterious effects on the biological environment. A particle is biocompatible if the substance is not toxic to the body or biological environment as intact particles or as dissociated ions (to an extent and at amounts that a sparingly soluble particle may dissociate in a given biological environment).

“Biodegradable” means that the polymer matrix can break down, degrade, or erode within a biological environment to non-toxic components after or while an active molecule has been or is being released to form smaller chemical species by enzymatic, chemical, physical, or other process.

An “inorganic element” according to the present invention, is a material comprising a metal component as well as mixtures, salts or hydrates thereof. The inorganic component may be selected from group of silica, alkaline metals, alkaline earth metals, transition metals, especially: zinc, calcium, magnesium, titanium, silver, aluminum or lanthanides, their salts, hydrates as well as combinations thereof. The inorganic element may be alkoxide, oxide, acetate, oxalate, ureate, or nitrate of the metal salt as well as hydrates thereof.

The term “active ingredient” comprises a drug, pharmaceutically active ingredient, biologically active ingredient or cosmetic active ingredient.

“Microparticles,” shall mean, particles having average particle diameter below 100 μm. In one of the preferred embodiments the particles are microparticles having particle diameter of less than about 10 μm.

“Nanoparticles,” shall mean, particles having average particle diameter below 2000 nm. In preferred embodiments the particles have an average particle diameter ranging from the group consisting of from about 1 nm to about 2000 nm, about 10 nm to about 200 nm, about 15 nm to about 150 nm.

As used herein, “average particle diameter” is used to refer to the size of particles in diameter, as measured by conventional particle size analyzers well known to those skilled in the art, such as sedimentation field flow fractionation, photon correlation spectroscopy, laser light scattering or dynamic light scattering technology and by using transmission electron microscope (TEM) or scanning electron microscope (SEM) or X-Ray diffraction (XRD). A convenient automated light scattering technique employs a Horiba LA laser light scattering particle size analyzer or similar device. Such analysis typically presents the volume fraction, normalized for frequency, of discrete sizes of particles including primary particles, aggregates and agglomerates. X-ray diffraction techniques are also widely used which determines the crystal size and shape and reveals information about the crystallographic structure, chemical composition and physical properties of materials.

The present invention also encompasses particles having mixtures of microparticles and nanoparticles. The particles are inclusive of “primary particles”; “secondary particles” and others thereof. In preferred embodiments of the invention particles may exist as loose aggregates which exhibit secondary particle size in diameter ranging from about 200 nm to 20 μm and primary particle size in diameters less than 200 nm, preferably less than 100 nm or less than 50 nm.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

Present invention relates to particles comprising inorganic element(s) for the delivery of active ingredients to human and animal tissues.

More specifically present invention relates to composition having particles comprising: inorganic element(s), one or more active ingredient(s) and optionally release rate modulating agent(s).

In a further embodiment invention relates to composition having particles comprising: inorganic element(s) in about 0.1% w/w to about 99.5% w/w; one or more active ingredient(s) in about 0.01% w/w to about 99.9% w/w and optionally release rate modulating agent(s) in about 0.001% w/w to about 75% w/w of the total weight.

The present invention also encompasses methods of preparation of compositions having particles comprising: inorganic element(s), one or more active ingredient(s) and optionally release rate modulating agent(s).

Methods of preparing particles especially nanoparticles are known in the art which can be broadly categorized in two classes, Top-down approaches and Bottom-up approaches. Top down approaches starts with bulk material and breaks it into smaller particles by mechanical, chemical or other form of energy to form nanoparticles whereas bottom-up approaches synthesize material from atomic or molecular species via chemical reactions, where precursor particles grow in size to form nanoparticles. Homogenization and milling comes in Top-down methods (principally used for drug nanoparticles) and precipitations, polymerization from monomers, desolvation/salting out/Solvent evaporation/solvent diffusion/solvent displacement for polymeric nanoparticles and sol-gel method are classified in category of bottom-up approaches. Other methods for forming drug nanoparticles include Aerosol flow reactor, microemulsion, supercritical fluid-based, media milling (Nanocrystal® Technology), high pressure homogenizers (Disso Cubes®) etc.

More specifically present invention relates to microparticles or nanoparticles, prepared by sol-gel method, for the delivery of active ingredients to human and animal tissues. The present invention relates to microparticles or nanoparticles, such as organic-inorganic hybrids, prepared by sol-gel synthesis, for the delivery of active ingredients to human and animal tissues. In a preferred embodiment the nanostructures of the invention may be produced by conventional sol-gel synthesis or any of its modifications known in the art. Such nanostructures will be biocompatible, produced at low temperatures and easily amenable to large scale production and are less expensive to manufacture.

In an embodiment the nanostructures of the present invention produced by sol-gel processes generally comprises of the following steps: preparation of a solution or suspension, of a precursor formed by a compound of the element (M) forming the oxide or alkoxide; hydrolysis (acid or base catalyzed), of the precursor, to form M-OH groups. The so obtained mixture, i.e. a solution or a colloidal suspension, is named sol; polycondensation of the M-OH or M-OR groups according to the reactions M-OH+M-OH→M-O-M+H₂O and M-OR+M-OH→M-O-M+ROH characterized by an increase of the liquid viscosity (gelation) and by the contemporaneous formation of a matrix called gel. The gel may be dried to a porous monolithic body or dried by a controlled solvent-evaporation, to produce xerogels, or by a solvent supercritical extraction to produce aerogels.

Alternatively, the process may involve the use of ‘template’ molecules during the sol-gel conversion, leading to the formation of ordered structures with well defined pore morphology. Examples of such structures are the mesoporous structures, micro-mesoporous structures etc. The template molecules may be inorganic or organic metal salts, small organic molecules, such as polyethylene glycol, long chain surfactant molecules, liquid crystal templates, room temperature ionic liquids etc.

In a specific embodiment sol gel process is performed by following steps: Dissolving active ingredient(s) in solvent to form solution (a), dissolving inorganic metal salt in solvent to form solution (b), dissolving a release rate modulating agent in solvent to form solution (c), wherein an alkali hydroxide solution is included in any of the step of ‘a’, ‘b’ or ‘c’ and mixing solutions (a), (b) and (c) to form a precipitate, drying the precipitate formed in step (d) to form a dry powder composition.

The drying of the precipitate can be done by, lyophilization, spray drying or spray freeze drying technique or combinations thereof.

The powder composition of the present invention may be applied as such or may be formulated into any other topical preparations like cream, ointment, lotion, gel, suspension and others by techniques known to a person skilled in the art.

In a further alternative method composition comprising inorganic element and optionally a release modulating agent can be prepared by performing the following steps: dissolving inorganic metal salt in a solvent to form solution (a), dissolving alkali metal hydroxide in a solvent to form solution (b), dissolving active ingredient and polymer in solvent to form solution (c), adding alkali metal hydroxide of solution (b) to solution (c) to form solution (d), adding inorganic metal salt of step (a) to prepared dispersion of step (d), stirring the resultant solution of (e) to a predetermined time and harvesting coarse aggregates by centrifugation and washing with water at least once to form and further dispersing the nanoparticles in solvent and finally preparing gel by thickening the dispersion.

It is to be understood that any modification in the type and manner of addition of the components in the steps of preparing the nanoparticles which is obvious to the person skilled in the art is also inclusive of the present invention.

According to this invention, alkali hydroxide can be present in a composition in an amount from about 5 to about 80% more preferably from about 15 to about 60% based on the final weight of the composition.

According to this invention, active ingredient is present in the composition in an amount in weight from about 0.01% to about 99.9% more preferably from about 0.03% to about 90% most preferably from about 1% to about 80% based on the final weight of the composition.

According to this invention, inorganic element is present in the composition in an amount in weight from about 0.1% to about 99.5% more preferably from 5% to about 95% and most preferably from 10% to about 80% based on the final weight of the composition.

According to this invention, rate modulating agent is present in the composition in an amount in weight from about 0.001% to about 75% more preferably from about 0.1 to about 60% and most preferably from 1% to about 50% based on the final weight of the composition.

According to this invention the alkali metal hydroxide is selected from but not limited to KOH, NaOH, LiOH, NH₄OH, Mg(OH)₂ hydrates thereof and combinations thereof.

According to the invention the solvent used in the sol-gel process is selected from a group consisting of water, C₁-C₅ alcohol including but not limited to methanol, ethanol, n-propanol, isopropanol and combinations thereof or organic species, including but not limited to, acetone, methylethyl ketone, tetrahydrofuran, benzene, toluene, o-xylene, m-xylene, p-xylene, mesitylene, diethyl ether, dichloromethane, chloroform, propylene glycol, triethanolamine and combinations thereof.

According to the invention the thickening agent used to formulate the compositions of the present invention is selected from but are not limited to xanthan gum, guar gum, locust bean gum and any other known excipients listed in the Handbook of Excipients.

Alternatively, the process may involve non-hydrolytic sol-gel process in the absence of water carried out by reacting alkylated metals or metal alkoxides with anhydrous organic acids, acid anhydrides or acid esters, or the like.

In preferred embodiments release modulating agent(s) in the composition are selected from the group of but not limited to natural polymers, synthetic polymers, semi synthetic polymers, lipids, waxes and natural or synthetic gums, polysaccharides, monosaccharide, sugars, salts, proteins, peptides, polypeptides and combinations thereof.

The natural, synthetic or semi-synthetic polymer especially biodegradable polymer or copolymer is selected from the group of but not limited to polyacrylates polymers, polyethylene oxide polymers, cellulose polymers, polyorthoesters, chitosan, polylactides, vinyl polymers and copolymers, alkylene oxide homopolymers polydioxanones, polyanhydrides, polycarbonates, polyesteramides, polyamides polyphosphazines, shellac derivatives and combinations thereof. A polymeric material as described herein may include a monomer, polymer or copolymer composition. Monomers are those capable of forming a macromolecule by a chemical reaction.

Suitable examples include a (methyl)acrylic monomer (e.g., methyl methacrylate, methyl acrylate and butyl acrylate).

In a specific preferred embodiment release rate modulating agent is a protein selected from gelatin, bovine serum albumin, human serum albumin and combinations thereof.

The release rate modulating agent can further be selected from the group comprising of cetyl pyridinium chloride, gelatin, casein, phosphatides, dextran, glycerol, gum acacia, cholesterol, tragacinth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β.-D-glucopyranoside; n-dodecyl β-D-maltoside; heptanoyl-N-methylglucamide; n-heptyl-.beta.-D-glucopyranoside; n-heptyl β-D-thioglucoside; n-hexyl β-D-glucopyranoside; nonanoyl-N-methylglucamide; n-noylβ.-D-glucopyranoside; octanoyl-N-methylglucamide; n-octyl-.β-D-glucopyranoside; octyl β.-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅ dimethyl hydroxyethyl ammonium chloride, C₁₂₋₁₅ dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide; lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecyldimethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar and combinations thereof.

The composition of the present invention may further contain hydrophilic solvents, lipophilic solvents, humectants/plasticizers, thickening polymers, surfactants/emulsifiers, fragrances, preservatives, chelating agents, UV absorbers/filters, antioxidants, keratolytic agents, dihydroxyacetone, penetration enhancers, dispersing agents or deagglomerating agents as well as mixtures thereof.

According to the invention the inorganic element(s) is selected from group comprising of silica, alkaline metals, alkaline earth metals, transition metals, especially zinc, calcium, magnesium, titanium, silver, aluminium, or lanthanides, their salts, hydrates, as well as combinations thereof.

In a preferred embodiment inorganic element is in the form of alkoxide, oxide, acetate, oxalate, ureate, or nitrate.

In a further preferred embodiment the inorganic element is selected from a group comprising of zinc oxide, calcium carbonate, calcium oxide, calcium hydroxide, calcium bicarbonate or combinations thereof.

In one embodiment of the invention, inorganic particles are prepared comprising the active ingredient(s), inorganic element and release rate modulating agents while in another embodiment the inorganic particles are prepared comprising the active ingredient(s) and inorganic element and then combined with organic release rate modulating agents such as acrylate polymers, such as polymethacrylates or polycyanoacrylates. The organic portion of the particles helps in controlling the release of active ingredients as well as affects the biodegradation and biodistribution of the system.

Incorporation of active ingredients into the sol-gel derived nanostructures may be done by any of the means known in the art. The incorporation may be done at any suitable stage during sol-gel synthesis, such as by co-condensation, if the ingredient can withstand the subsequent steps in the synthesis. The systems can be prepared by molecular imprinting or impregnation of the active ingredients within the nanostructures. Alternatively the incorporation may be done by loading after the basic metal or organo-metal hybrid structure has been prepared.

In an embodiment of the invention the particles may be in the form of nanoparticles, nanospheres, nanorods, nanotubes, monolithic systems, indented systems, aggregates or combinations thereof. They may also be processed to form ordered materials such as mesoporous, microporous or macroporous structures. The particles may degrade and release the active ingredients by surface erosion or biodegradation in presence of physiological fluid.

In preferred embodiments the composition is useful for topical application to the skin, to a mucosal surface like rectal, vaginal, surface of the eye, nasal passages, and mouth and lip area or the external ear.

The compositions of the present invention are formulated as cream, lotion, gel, paste, powder, sprays, foam, roll-ons, oils, patches, suspensions, ointment, deodorant or aerosols.

In an embodiment of the invention, the invention also relates to a kit comprising a delivery device; the composition having particles comprising: inorganic element(s), one or more active ingredient(s), optionally release rate modulating agent(s) and instructions for its use; for the delivery of the composition to topical or mucosal surfaces. The delivery device comprises a pressurized or non-pressurized dispensing device or applicator or mechanical device, which delivers the composition to the topical or mucosal surfaces. In a preferred embodiment the delivery device is capable of delivering metered dose of the composition to the topical or mucosal surfaces.

In an embodiment of the invention non-pressurized dispensing devices can be selected from the group of but are not limited to hand held squeeze containers, tubes, powder dispensing containers, roll-ons.

In a further embodiment of the invention delivery device can also be an applicator selected from but not limited to a brush, a spatula or a spoon.

In another aspect, the compositions can be delivered in a solid form to the topical or mucosal surface. In one embodiment, the solid form can be dry powder form which is applied with the help of a dispensing device such as a metered dose dispensing container or a roll-on stick or the powder dosage form can be simply applied and rubbed by hand by the patient/subject at the site of use.

In another embodiment of the invention the formulations can be dispensed by pressurized devices by spraying. In one embodiment, the compositions are sprayed as a dry powder from a pressurized can, or less preferably from a hand-pumped container. In a pressurized can, any medically approved propellant is potentially suitable, which includes alkanes such as propane and butane, and approved hydrofluoroalkanes, such as tetrafluoroethane (HFA 134a) and heptafluoropropane (HFA 227). Optionally and preferably, the preparation to be sprayed contains enhancers. The formulation may contain a surfactant to maintain the various ingredients in a single phase, or as a two-phase preparation that will re-emulsify upon brief shaking.

The spray may contain other sprayable components. These may include oily or occlusive materials, such as vegetable oil, or a polymer that is soluble in the propellant but which precipitates on the skin as the solvent evaporates. The spray solution in the can may also contain surfactants, to keep the components mixed. It may also contain combinations of surfactants and polymers that will foam on emergence from the aerosol can. The foam will carry the compositions comprising active ingredients and inorganic elements, and optionally enhancers, and will deposit these active ingredients on the skin in a non-running, well-localized manner. The foam will preferably collapse, immediately or gradually, and preferably upon contact with tissue exudates, thereby delivering the compositions to the tissue surface.

Any pharmaceutically acceptable hydrocarbon, CFC or HFA propellant can be used in the formulations. The preferred propellant of an aerosol formulation is a HFA (hydrofluoroalkane, also known as hydrofluorocarbon, HFC), such as HFA 134a (tetrafluoroethane) or HFA 227 (heptafluoropropane) or other HFA approved for medical use. The HFAs have a much lower ozone destroying potential than chlorofluorocarbons (CFCs) and are currently approved as propellants. They are non-flammable, unlike the alkane propellants, such as propane and butane. In the literature, HFAs are often used with irritating and/or flammable co-solvent materials, such as ethanol and other lower alcohols, to reduce pressure. A co-solvent is not necessary in the formulations. The HFA is charged to the spray container so as to form about 10% to about 50% of the final weight of the container's contents, more preferably at about 15% to about 40%, still more preferably at about 20% to about 35%. The emollient preferably dissolves or co-emulsifies in the balsam/castor oil/surfactant material when in combination with the propellant, and does not precipitate out or otherwise phase separate when with the propellant at room temperature (ca. 20° C.), or preferably at 15° C. or below.

The spray can is conventional, and preferably is aluminum with an inner coating of epoxy or other passivating lining. A preferred feature of the spray can is a multi-angle spray head/dispenser, which can dispense the formulation from angles other than purely upright.

According to the invention the composition comprises the active ingredient(s) wherein the active ingredient is selected from and is not limited to antibiotics, antiviral agents, anti-fungals, analgesics, anorexics, antipsoriatics and acne treatment agents, anti herpes agents, antihelminthics, antiarthritics, antiasthmatic agents, anticonvulsants, antidepressants, antidiabetic agents, antidiarrheals, antihistamines, antiinflammatory agents, antimigraine preparations, antinauseants, antiandrogens, antisyphilictic agents, antineoplastics, antiparkinsonism drugs, antipfuritics, antipsychotics, antipyretics, antispasmodics, anticholinergics, sympathomimetics, xanthine derivatives, cardiovascular preparations including potassium and calcium channel blockers, beta-blockers, alpha-blockers, and antiarrhythmics, antihypertensives, diuretics and antidiuretics, vasodilators including general coronary, peripheral and cerebral, central nervous system stimulants, vasoconstrictors, cough and cold preparations, including decongestants, hormones such as testosterone, estradiol and other steroids, including corticosteroids, hypnotics, immunosuppressives, muscle relaxants, parasympatholytics, psychostimulants, dermatitis herpetoformis suppressants, topical protectants, mosquito repellants, anti-lice agents, sedatives, tranquilizers, macromolecules such as proteins, polypeptides, polysaccharides, vaccines, antigens, antibodies and combinations thereof.

In further embodiments the active ingredient(s) of the composition is useful for cosmetic preparations, selected from the group of but not limited to antiageing agents, sunblocking agents, antiwrinkle agents, moisturizing agents, anti-dandruff agents especially selenium sulfide, vitamins, saccharides, oligosaccharides, hydrolysed or non-hydrolysed, modified or unmodified polysaccharides, amino acids, oligopeptides, peptides, hydrolysed or non-hydrolysed, polyamino acids, enzymes, branched or unbranched fatty acids and fatty alcohols, animal, plant or mineral waxes, ceramides and pseudoceramides, hydroxylated organic acids, antioxidants and free-radical scavengers, chelating agents, seborrhoea regulators, calmants, cationic surfactants, cationic polymers, amphoteric polymers, organomodified silicones, mineral, plant or animal oils, polyisobutenes and polyα.-olefins), fatty esters, anionic polymers in dissolved or dispersed form, nonionic polymers in dissolved or dispersed form, reducing agents, hair dyes or pigments, antioxidants, free radical scavengers, melanoregulators, tanning accelerators, depigmenting agents, skin-coloring agents, liporegulators, thinning agents, antiseborrhoeic agents, anti-UV agents, keratolytic agents, refreshing agents, cicatrizing agents, vascular protectors, antiperspirants, deodorants, skin conditioners, immunomodulators, nutrients and essential oils and perfumes, substance having a hair-care activity, agents for combating hair loss, hair dyes, hair bleaches, reducing agents for permanent waves, hair conditioners, nutrients or combinations thereof.

In a further embodiment the active ingredient in the composition is a peptide having molecular weight less than 100 kilo daltons selected from the group of but not limited to hair growth promoting actin binding peptides, RNA III Inhibiting peptides, cosmetically active peptides and peptide based colorants

Some specific examples of the active ingredients in the class of antiviral agents, antifungal agents, antibacterial agents, antialopecia agents, antiacne agents, antipsoriatics agents and immunosuppressants used in the composition are listed.

Antiviral agent is selected from group of but not limited to acyclovir, ganciclovir, famciclovir, foscamet, inosine-(dimepranol-4-acetamidobenzoate), valganciclovir, valacyclovir, cidofovir, brivudin, antiretroviral active ingredients (nucleoside analog reverse-transcriptase inhibitors and derivatives) such as lamivudine, zalcitabine, didanosine, zidovudin, tenofovir, stavudin, abacavir, non-nucleoside analog reverse-transcriptase inhibitors such as amprenavir, indinavir, saquinavir, lopinavir, ritonavir, nelfinavir, amantadine, ribavirin, zanamivir, oseltamivir as well as any combinations thereof.

Antifungal agent is selected from but not limited to allylamines (ammolfine, butenafine, naftifine, terbinafine), azoles (ketoconazole, fluconazole, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole), polyenes (nystatin, pimaricin, amphotericin B), pyrimidines (flucytosine), tetraenes (natamycin), thiocarbamates (tolnaftate), sulfonamides (mafenide, dapsone), glucan synthesis inhibitors (caspofungin), benzoic acid compounds, complexes and derivatives thereof (actofunicone) and other systemic or mucosal (griseofulvin, potassium iodide, Gentian Violet) and topical drugs (ciclopirox, ciclopirox olamine, haloprogin, undecylenate, silver sulfadiazine, undecylenic acid, undecylenic alkanolamide, Carbol-Fuchsin) as well as any combinations thereof.

Antibacterial agents are selected from but not limited to aclacinomycin, actinomycin, anthramycin, azaserine, azithromycin, bleomycin, cuctinomycin, carubicin, carzinophilin, chromomycines, clindamycin, ductinomycin, daunorubicin, 6-diazo-5-oxn-1-norieucin, doxorubicin, epirubicin, mitomycins, mycophenolsaure, mogalumycin, olivomycin, peplomycin, plicamycin, porfiromycin, puromycin, streptonigrin, streptozocin, tubercidin, ubenimex, zinostatin, zorubicin, aminoglycosides, polyenes, macrolid-antibiotics derivatives and combinations thereof.

Antialopecia agent is selected from but not limited to the group comprising minoxidil, cioteronel, diphencyprone and finasteride and combinations thereof.

Antiacne agent is selected from but not limited to the group comprising retinoids such as tertionin, isotretionin, adapalene, algestone, acetophenide, azelaic acid, benzoyl peroxide, cioteronel, cyproterone, motrtinide, resorcinol, tazarotene, tioxolone as well as any combinations thereof.

Antipsoriatics agent is selected from but not limited to the group comprising dithranol, acitretin, ammonium salicylate, anthralin, 6-azauridine, bergapten, calcipotriene, chrysarobin, etritrenate, lonapalene, maxacalcitol, pyrogallol, tacalcitol and tazarotene as well as any combinations thereof.

Immunosuppressant is selected from but not limited to the group comprising tacrolimus, cyclosporine, sirolimus, alemtuzumab, azathioprine, basiliximab, brequinar, Daclizumab, gusperimus, 6-mercaptopurine, mizoribine, muromonab CD3, pimecrolimus, rapamycin and combinations thereof.

Synthetic mosquito repellent is selected from but not limited to the group comprising N,N-diethyl-meta-toluamide (DEET), NN Diethyl Benzamide, 2,5-dimethyl-2,5-hexanediolbenzil, benzyl benzoate, 2,3,4,5-bis(butyl-2-ene)tetrahydrofurfural (MGK RepellentII), butoxypolypropylene glycol, N-butylacetanilide, normal-butyl-6,6-dimethyl-5,6-dihydro-1,4-pyrone-2-carboxylate (Indalone), dibutyl adipate, dibutyl phthalate, di-normal-butyl succinate (Tabatrex), dimethyl carbate (endo,endo)-dimethyl bicyclo[2.2.1]hept-5-ene-2,3-dicarboxylate), dimethyl phthalate, 2-ethyl-2-butyl-1,3-propanediol, 2-ethyl-1,3-hexanediol (Rutgers 612), di-normal-propyl isocinchomeronate (MGK Repellent326), 2-phenylcyclohexanol, p-methane-3,8-diol, and normal-propyl N,N-diethylsuccinamate and derivatives or combinations thereof or natural insect repellents selected from group of Dihydronepetalactone, Eucalyptus-derived p-menthan-3,8-diol (PMD) repellent, E-9-octadecenoic acid-derived compounds, extracts from limonene, citronella, eugenol, (+) eucamalol (1), (−)-1-epi-eucamalol, or a crude extract from plants such as Eucalyptus maculata, Vitex rotundifolia, or Cymbopogan, maltitol compound, peppermint oil, cinnamon oil, and nepetalaclone oil, Azadirachitin, other neem derived compounds and combinations thereof.

In one of preferred embodiments the composition of the present invention is a dry powder composition of acyclovir for topical or mucosal application wherein the acyclovir is present in the dose range from about 1% to about 10%.

Acyclovir is an antiviral drug, which is used to treat infections caused by herpes viruses. Illnesses caused by herpes viruses include genital herpes, cold sores on the face or lips, shingles, and chicken pox. Topical acyclovir is available as a cream and an ointment to apply to the skin. Acyclovir cream is usually applied five times a day for 4 days. Acyclovir ointment is usually applied six times a day (usually 3 hours apart) for 7 days. Thus the presently marketed preparations require frequent applications and is not patient compliant. The dry powder compositions of the present invention comprising acyclovir along with inorganic element and optionally a release modulating agent allows for reduced frequency of application due to its ability to be retained in upper layers of the skin.

In another preferred embodiments the composition comprises terbinafine present in the dosage range of from about 1% to about 10%.

Terbinafine is an antifungal agent, which is used to treat skin infections such as athlete's foot, jock itch, and ringworm infections. Terbinafine is mainly effective on the dermatophytes group of fungi. As a 1% cream it is used for superficial skin infections such as jock itch (Tinea cruris), athlete's foot (Tinea pedis) and other types of ringworm. It is available as topical cream, gel, ointment, solution and sprays. The dry powder compositions of the present invention comprising terbinafine along with inorganic element and optionally a release modulating agent allows for reduced frequency of application, due to its ability to be retained in upper layers of the skin. It is easy to apply and is non irritating and non visible on application at the site of action.

In one of the preferred embodiments the composition comprises the active ingredient(s) wherein the active ingredient is Clindamycin. In a further embodiment clindamycin is present in the dosage range of from about 1% to about 10%.

Clindamycin, an antibiotic is used to treat infections of the respiratory tract, skin, pelvis, vagina, and abdomen. Topical application of clindamycin phosphate is used to treat moderate to severe acne. This medication is marketed under various trade names including Dalacin® (Pfizer), Cleocin® (Pfizer) and Evoclin® (Connetics)—in a foam delivery system. The dry powder compositions of the present invention comprising clindamycin along with inorganic element and optionally a release modulating agent allows for reduced frequency of application due to its ability to be retained in upper layers of the skin.

In one of the preferred embodiment the composition comprises the active ingredient(s) wherein the active ingredient is a Mosquito repellents especially Meta-N,N-diethyl toluamide or NN Diethyl Benzamide. In a further embodiment Meta-N,N-diethyl toluamide or NN Diethyl Benzamide is present in the dosage range of from about 1% to about 95%. Meta-N,N-diethyl toluamide, abbreviated DEET, is an insect-repellent chemical. It is intended to be applied to the skin or to clothing, and is primarily used to protect against insect bites. In particular, DEET protects against tick bites (which transmit Lyme disease) and mosquito bites (which transmit dengue fever, West Nile virus, Eastern Equine Encephalitis (EEE), and malaria).

NN Diethyl Benzamide is commonly used topical insect repellent in 12% concentration. Its ability to protect against mosquitoes is well documented. Dermal absorption studies done reveals that there is no absorption of the active ingredient in the blood plasma, thereby concurring that it is absolutely safe on human skin.

The dry powder compositions of the present invention comprising mosquito repellents along with inorganic element and optionally a release modulating agent allows a reduced frequency of application due to its ability to be retained in upper layers of the skin.

In one of the embodiment, the composition comprises the active ingredient(s) wherein the active ingredient is hair growth hormone. In a further embodiment hair growth promoting actin binding peptide homologous to Thymosin β₄ is present in the dosage range from about 0.001% to about 20%.

In one of the preferred embodiments of the invention the compositions comprises a minimal fragment of the thymosin-β₄ sequence that is associated with hair growth, for example the “T-3” fragment (residues 17-23) or the shorter actin binding sequence (residues 17-22). These fragments, or peptides comprising these sequences, are used to promote hair growth on humans and other animals. In some embodiments, a peptide comprising one or more of these sequences is applied topically to the area to be treated. In other embodiments, the peptide is applied in conjunction with additional compositions, including but not limited to antimicrobials, antiparasitics, skin and/or hair conditioners, soaps, emollients, and other suitable compositions. It is also contemplated that variants or homologues of these sequences (e.g., with conservative and/or non-conservative amino acid changes) will find use in promoting hair growth. Alternatively, the peptides include full-length thymosin-β₄ sequences, with one or more conservative substitutions outside of the actin binding sequence (residues 17-22). Polymers of actin-binding polypeptide moieties (for example dimers and trimers of thymosin-(β₄ or a peptide containing the sequence of amino acids 17-22 of thymosin-β₄) can also show enhanced hair growth activity, as can any of several fusion molecules bonded to an actin-binding moiety. Thus, an actin-binding moiety useful in the present disclosure may be expressed in many ways, including as part of a larger peptide, as part of a fusion molecule, or in a polymer or combinations thereof. The dry powder compositions of the present invention comprising hair growth promoting actin binding peptide homologous to thymosin β₄ along with inorganic element and optionally a release modulating agent uses a reduced frequency of application due to its ability to be retained in layers of skin such as stratum corneum and epidermis and dermis and combinations thereof. It is very easy to apply, is non irritating and non visible on application thus offering better patient compliance.

An aspect of the invention relates to a composition, especially in the dry powder form, wherein it is possible to incorporate higher percentage of active ingredient in the composition, even as high as about 5% w/w to about 80% w/w. Such composition when applied at the site of action like skin was also found to be non-visible, when tested in animals.

According to the compositions of this invention since the active ingredients is retained in the skin for a longer period of time at the site of action; it is required less frequently compared to the approved dosages. Thus in exemplary embodiment the composition is applied two times a day, once a day, once in two days, thrice a week, twice a week and once in a week based on the type of the active ingredient used.

The studies of in-vitro skin permeation are the most common experimental set-ups for the control of dermatological formulations. It has been carried out using a wide variety of experimental protocols dependent on the research group, the substances in study and the purpose of the substance or formulation applied to the skin. The in-vitro methods involve the diffusion measurement of substances through the skin, bioengineered, various skin layers, or artificial membranes to a receptor fluid assembled in a diffusion cell, which can be static or flow-through.

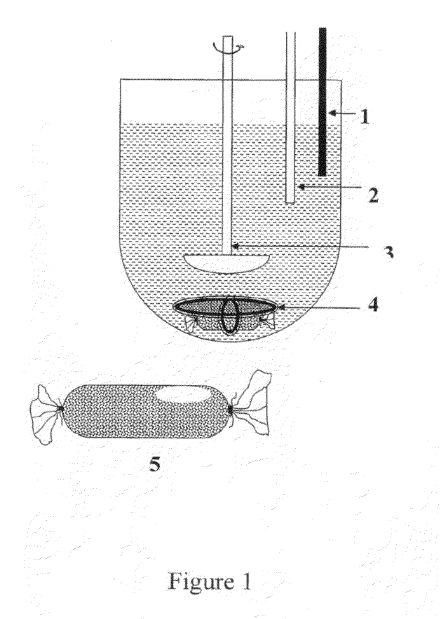

The in-vitro studies of the dissolution of the particles of the composition were performed in a specially designed Drug Release apparatus, the apparatus comprises a dialysis bag with specially treated membrane. Treatment of Dialysis Membrane (25 mm×16 mm; Sigma-Aldrich; Mol Wt cut off 12.4 kDa) was done in the following manner. Glycerol is used in the dialysis tubing as a humectant. It was removed by washing the tubing in running water for 3-4 hours. Removal of sulfur compounds was accomplished by treating the tubing with a 0.3% (w/v) solution of sodium sulfide at 80 deg. C. for 1 minute. Tubing was washed with hot water (60 deg. C.) for 2 minutes, followed by acidification with a 0.2% (v/v) solution of sulfuric acid, and then rinsed with hot water to remove the acid. This treated dialysis tubing was used to prepare dialysis bag by tying one end of the tube. Sample was poured in the tube followed by closing the other end used thread knot. Due to air sac this dialysis bag floats on the surface of the media. Perforated metal disc were used to sink dialysis bag in release media. The apparatus is illustrated in FIG. 1.

The compositions of the present invention provide controlled release profile of the active ingredient released from the group of monophasic, biphasic or multiphasic release profile. In a preferred embodiment not more than 60% of the total amount of active ingredient is released within 2 hours and not less than 75% of the active ingredient is released within 14 hours when the composition is subjected to in-vitro dissolution studies. In an embodiment the studies was conducted using modified USP paddle method in pH 4.5 acetate buffer.

Two tape stripping studies were conducted, in rats and in guinea pigs, in order to know the dermatokinetics of different formulations in the different layers of skin which includes stratum corneum, epidermis and dermis. It has been shown that by stripping the stratum corneum from a small area of the skin with repeated application and removal of cellophane tape to the same location one can easily collect arbitrary quantities of interstitial fluid, which can then be assayed for a number of analytes of interest. Experiment was done by applying the tape onto a selected area of skin to adhere the tape to the selected skin area; stripping the tape off the selected skin area to obtain a sample representative of an outer stratum corneum layer of the skin, the sample adhering to the tape so as to have exposed skin constituents.

In experiments conducted on wistar rats and guinea pigs, hairs were depilated from the back (around dorsal region) of the animals, between the fore and hind limbs. Dorsal sides of the animals were marked in 3×3 cm² areas. Test (present invention) and Reference (Commercially available) formulations were applied with a contact time of 2 hours. Adhesive tape (Transpore 3M) was used for stripping procedure (exposed skin site was retained with adhesive tape for 1 minute and peeled off from the site using a forceps at constant force with 30-45 degree angle in order to minimize the effect of the peeling force on removal efficiency). Ten stripping were performed at each time points 6 and 24 hours in guinea pigs and at 0, 3, 6, 14 and 24 hrs in rats from the exposed region after the application. Untreated group was also exposed to similar stripping procedure and were analyzed by HPLC for the estimation of the active ingredient. The results for an embodiment are discussed in table 8 and 9.

In another embodiment composition according to the present invention further includes occlusive patches for the prevention of particles escaping to the exterior of the application area.

Thus present invention addresses the need in the art for a composition of active molecules, especially for better local delivery of active ingredients on the surface of skin or mucosal surfaces. This technology for the delivery of active ingredients offers advantages like ease of use, better retention at site of action, effective rates of absorption, controlled release over a desired period of time, dose reduction and better cosmetic and aesthetic compliance. Besides that the compositions of the present invention are not irritating to skin and not visible when applied to skin or mucosal surfaces, they are also easy to apply, and have a better patient compliance.

The following Examples are intended to further illustrate certain particularly preferred embodiments of the invention and are not intended to limit the scope of the invention in any way.

### Example 1

TABLE 1

| Ingredients | A | | B | | C | | D | |
|---|---|---|---|---|---|---|---|---|
| Terbinafine HCl | 1.5 | g | 1.5 | g | 1.5 | g | 1.5 | g |
| Hydroxypropyl | 300 | mg | 300 | mg | 300.0 | mg | — | |
| Cellulose (HPC) | | | | | | | | |
| Zinc nitrate | 22.31 | g | — | | 22.3 | g | 22.3 | g |
| hexahydrate | | | | | | | | |
| Zinc acetate | — | | 25.5 | g | — | | — | |
| dihydrate | | | | | | | | |
| Potassium | 8.4 | g | 15.1 | g (in 50 g | 8.4 | g | 8.4 | g |
| hydroxide | | | | Methanol) | | | | |
| Methanol | 75 | g | 100 | g | 60.0 | g | 60.0 | g |

| | | | | | | | |
|---|---|---|---|---|---|---|---|
| Water | 75 | g | — | 90.0 | g | 90.0 | g |

### Method for ‘A’

The metal oxide nanoparticles were synthesized by first dissolving zinc nitrate in water 50 g and in another solution, potassium hydroxide was dissolved in water 25 g; following these two steps, separately Terbinafine HCl and hydroxypropyl cellulose were dissolved in methanol to form a solution ‘A’. Potassium hydroxide solution prepared earlier was added drop wise to the solution ‘A’ under continuous stirring which continued for about 20 minutes to form dispersion solution ‘B’. Zinc nitrate solution prepared earlier was added to the dispersed solution ‘B’ in drop wise manner. The resultant solution was stirred and centrifuged followed by washing three times with water to give white course aggregates (nanoparticles). These nanoparticles were further dispersed in a mixture of propylene glycol, water and triethanolamine (in ratio 40:45:15) to form dispersion Mixture ‘C’. Finally a gel was prepared from the dispersion mixture using suitable polymer, like xanthan gum.

### Method for ‘B’

Terbinafine HCl and hydroxypropyl cellulose were dissolved in methanol to form a drug polymer solution. In another step potassium hydroxide was dissolved in methanol followed by keeping the solution in an ice bath. In a further critidal step zinc acetate was added at a high temperature to the drug polymer solution prepared earlier. The resultant Solution ‘A’ containing drug, polymer and zinc acetate was added to potassium hydroxide solution kept in ice bath under stirring, to form the nanoparticles, which were washed three times with methanol. Nanoparticles obtained were further dispersed in a mixture of propylene glycol, water and triethanolamine (in ratio 40:45:15) to form a dispersion mixture. Finally a gel was prepared from the dispersion mixture using suitable polymer, like xanthan gum,

### Methods for ‘C’ and ‘D’

For making composition ‘C’ Terbinafine, HPC and zinc nitrate were taken and for making composition ‘D’ Terbinafine and zinc nitrate were taken and were dissolved in methanol to form solution ‘A’ and potassium hydroxide was dissolved in water to form solution ‘B’. Solution of ‘step B’ was then added to solution of ‘step A’ at a controlled rate (0.2-0.5 ml/min) under continuous stirring at 500 rpm. The dispersion thus obtained was lyophilized to obtain fine powder.

### Assay

Each of the above compositions was analyzed for drug content (total drug). Weighed quantity of particles containing drug was dissolved in 1N HCl followed by dilution with methanol. Drug was measured against the standard using HPLC. In case of composition ‘C’, each gram of powder contained 65.4 mg of Terbinafine HCl.

### Entrapment Efficiency

Entrapment efficiency of the lyophilized powder of composition ‘C’ was measured. The powder was dispersed in pH 4.5 acetate buffer and centrifuged at 10,000 rpm for 10 min at 25° C. Amount of drug in supernatant buffer was measured, which is the unentrapped drug. Entrapment efficiency (ER) was calculated by using equation 1

% EE=((Total Drug−unentrapped drug)/Total Drug)×100 (Equation 1)

The results indicated 99.70% entrapment efficiency

### Particle Size Data

Particle size distribution for the composition ‘C’ was measured at the dispersion stage. The obtained dispersion was diluted with purified water (1 to 10) and sonicated for 10 minutes using probe sonicator (0.8 Cycle; 60% amplitude) with an ice bath. Particle size distribution was measured using Horiba Partica LA-950 (Fraction Cell). Table 2 below gives the particle size data

TABLE 2

| | | Particle size |
|---|---|---|
| S. No. | Parameter | (μm)* |
| 1 | Mean size | 10.45 |
| 2 | D₁₀ | 5.95 |
| 3 | D₅₀ | 9.95 |
| 4 | D₉₀ | 15.58 |
| 5 | D₉₅ | 17.51 |

*Aggregates were observed in the dispersion; hence this is representation of secondary particle size

The crystal size of the particles was also determined using X-ray diffraction technique and was found to be 31.88 nm (upper limit of 49.23 nm; lower limit of 28.34 nm).

### Example 2

TABLE 3

| Ingredients | E | | F | | G | | H | | I | |
|---|---|---|---|---|---|---|---|---|---|---|
| Acyclovir | 1.5 | g | 1.5 | g | 1.5 | g | 1.5 | g | 1.5 | g |
| Gum acacia | — | | — | | 4.0 | g | — | | — | |
| Mannitol | — | | 44.0 | g | — | | — | | — | |
| Potassium | 8.4 | g | — | | — | | — | | — | |
| Hydroxide | | | | | | | | | | |
| (KOH) | | | | | | | | | | |
| Sodium | — | | 4.0 | g | 4.0 | g | 4.0 | g | 4.0 | g |
| Hydroxide | | | | | | | | | | |
| (NaOH) | | | | | | | | | | |
| Zinc Nitrate | 22.3 | g | 14.87 | g | 14.87 | g | 14.87 | g | 14.87 | g |
| Hexahydrate | | | | | | | | | | |
| Water | 175 | g | 200 | g | 175 | g | 150.0 | g | 150.0 | g |

| | | | | | | | | | |
|---|---|---|---|---|---|---|---|---|---|
| HPC-L | 300 | mg | 300.0 | mg | 300.0 | mg | 300.0 | mg | — |

### Method for E

Acyclovir along with, potassium hydroxide was dissolved in 50 g of water. Then, zinc nitrate was dissolved in 50 g of water. Separately HPC was dissolved in 75 g water. Further, simultaneous mixing of all three solutions under stirring results in a white precipitate. The precipitate was lyophilized and resulted in a white powder.

### Method for F

Mannitol was dissolved in 100 g water to form Solution ‘A’ Separately acyclovir and NaOH were dissolved in 50 g water to form Solution ‘B’. In another step HPC and Zinc nitrate were dissolved in 50 g water to form Solution ‘C’. Simultaneously drug solution of Solution ‘B’ and zinc nitrate solution ‘C’ were added to mannitol solution ‘A’ under stirring at 500 rpm using mechanical stirrer. The dispersion was diluted (four times) with purified water. Further the dispersion was spray dried as well as lyophilized to obtain fine powder.

### Methods for G, H and I

Gum acacia as shown in Table 2 Method ‘G’ is dissolved in purified water (75.0 g) to form solution ‘A’ and HPC and zinc nitrate are dissolved in purified water 50.0 g to form solution ‘B’. In another step the active molecule acyclovir is dissolved in sodium hydroxide to form solution ‘C’. Further solutions ‘A, B, C’ are mixed for composition ‘G’ at a controlled rate (0.2-0.5 ml/min) under continuous stirring at 500 rpm in to form a dispersion and solutions ‘B, C’ are mixed at a controlled rate (0.2-0.5 ml/min) under continuous stirring at 500 rpm in composition ‘H’ to form dispersion. Composition ‘I’ is made the same way as composition ‘H’ except that HPC is not used. Further the dispersion obtained is lyophilized to form dry powder acyclovir formulation.

Assay: Each of the above compositions was analyzed for drug content (total drug). Weighed quantity of particles containing drug was dissolved in 0.1N HCl and measured against the standard using HPLC. In case of composition ‘G’, each grain of powder contains 80.70 mg of acyclovir

### Entrapment Efficiency:

Entrapment efficiency of lyophilized powder of composition ‘G’ was measured. The powder was dispersed in pH 4.5 acetate buffer and centrifuged at 10,000 rpm for 10 min at 25° C. Amount of unentrapped drug in supernatant buffer was measured, which is the unentrapped drug. Entrapment efficiency was calculated by using equation 1. The results indicated 91.96% entrapment efficiency.

### Particle Size Data

Particle size distribution of acyclovir composition ‘G’ was measured at the dispersion stage. The obtained dispersion was diluted with purified (1 to 10) and sonicated for 10 minutes using probe sonicator (0.8 Cycle; 60% amplitude) with an ice bath. Particle size distribution was measured using Horiba Partica LA-950 (Fraction Cell). Table 4 below gives the particle size data

TABLE 4

| S. No. | Parameter | Particle size (nm) |
|---|---|---|
| 1 | Mean size | 107.3 |
| 2 | D₁₀ | 90.6 |
| 3 | D₅₀ | 107.3 |
| 4 | D₉₀ | 125.8 |
| 5 | D₉₅ | 129.3 |

The crystal size of the particles was also determined, using X-ray diffraction technique and was found to be 67.62 nm (upper limit of 104.44 nm; lower limit of 60.11 nm).

### Drug Release

The parameters used for drug release studies in USP dissolution apparatus and its process is described below:

### Drug Release Media/Volume: pH 4.5 Acetate Buffer/500 ml

Method: USP2 paddles/50 rpm over perforated metal disc tide to dialysis bag
Dialysis bag details: Length: Total 8 cm (1.5 cm for thread knots on both sides; Effective area for diffusion 5 cm).
Weight of powder in bag: 506.7 mg
Volume of media in bag: 5.0 ml
Amount of drug/unit: 40.9 mg

### Temperature: 370.5° C.

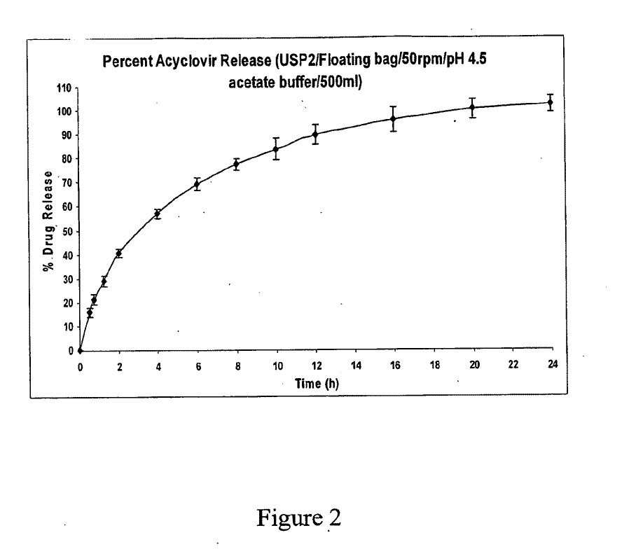

Treated dialysis tubing was used to prepare dialysis bag by tying one end of the tube. Sample was poured in the tube followed by closing the other end used thread knot. Dialysis bag was tied to perforated metal disc, which was in turn placed at the bottom of the vessel containing release media set at 37±0.5° C. Sampling: 10 ml with replacement. Results of the experiment are illustrated in FIG. 2.

### Example 3

TABLE 5

| S. No. | Ingredient | J | | K | | L | |
|---|---|---|---|---|---|---|---|
| 1 | Gum acacia | 4.0 | g | — | | — | |
| 2 | Purified water | 75.0 | g | — | | — | |
| 3 | Hydroxy propyl cellulose | 300.0 | mg | 300.0 | mg | — | |
| 4 | Zinc nitrate hexahydrate | 14.87 | g | 14.87 | g | 14.87 | g |
| 5 | Clindamycin phosphate | 1.0 | g | 1.0 | g | 1.0 | g |
| 6 | Purified water | 50.0 | g | 75.0 | g | 75.0 | g |
| 7 | Sodium hydroxide | 4.0 | g | 4.0 | g | 4.0 | g |
| 8 | Purified water | 50.0 | g | 75.0 | g | 75.0 | g |

### Methods for J, K and L

Gum acacia was dissolved in purified water 75.0 g to form solution ‘A’ and hydroxy propyl cellulose and Zinc nitrate were dissolved in purified water to form solution ‘B’. In another step the active molecule Clindamycin is dissolved in alkali solution of sodium Hydroxide to form Solution C. Further solutions A, B, C (Method ‘J’) or B, C (Method ‘K’) are mixed at a controlled rate (0.2-0.5 ml/min) under continuous stirring to form dispersion. This dispersion was lyophilized to form dry powder clindamycin formulation. In a similar method ‘L’ zinc nitrate was dissolved in purified water to form solution ‘A’ and clindamycin was dissolved in alkali to form solution ‘B’. Further solutions A, B are mixed at a controlled rate (0.2-0.5 ml/min) under continuous stirring to form dispersion. This dispersion was lyophilized to form dry powder clindamycin formulation.

### Example 4

TABLE 6

| Ingredient | M | | N | | O | | P | | Q | | R | |
|---|---|---|---|---|---|---|---|---|---|---|---|---|
| Thymosin β4 | 10 | mg | 10 | mg | 10 | mg | 10 | mg | 10 | mg | 10 | mg |
| Zinc nitrate hexahydrate | 14.87 | g | 14.87 | g | 14.87 | g | 14.87 | g | 14.87 | g | 14.87 | g |
| Hydroxy propyl cellulose | — | | — | | — | | 300.0 | mg | 300.0 | mg | — | |
| Purified water | 50.0 | g | 50.0 | g | 50.0 | g | 50.0 | g | 50.0 | g | 50.0 | g |
| Bovine serum albumin | 5.0 | g | 5.0 | g | — | | — | | — | | — | |
| Gum Acacia | 4.0 | g | — | | 4.0 | g | 4.0 | g | — | | — | |
| Purified Water | 75.0 | g | 75.0 | g | 75.0 | g | 75.0 | g | 75.0 | g | 75.0 | g |
| Sodium hydroxide | 4.0 | g | 4.0 | g | 4.0 | g | 4.0 | g | 4.0 | g | 4.0 | g |
| Purified Water | 50.0 | g | 50.0 | g | 50.0 | g | 50.0 | g | 50.0 | g | 50.0 | g |

### Methods for ‘M’, ‘N’, ‘O’, ‘P’, ‘Q’ and ‘R’

Thymosin β4 peptide was dissolved in zinc nitrate, HPC and in water to form solution ‘A’. I in another solution dissolve bovine serum albumin and gum acacia in water to form solution ‘B’. In a further step sodium hydroxide was dissolved in water to form solution ‘C’. Solutions ‘A’ and ‘C’ were added under continuous stirring at 500 rpm to obtain dispersion. The resultant dispersion was further lyophilized to obtain dry powder. The concentrations of components of compositions ‘M’, ‘N’, and ‘O’, ‘P’, ‘Q and R’ are described in table 6.

### Example 5

TABLE 7

| Ingredient | S | | T | | U | | V | |
|---|---|---|---|---|---|---|---|---|
| N-N diethyl benzamide | — | | 10.0 | g | — | | 10.0 | g |
| N-N diethyl-meta- | 10.0 | g | — | | 10.0 | g | — | |
| toluamide | | | | | | | | |
| Hydroxy propyl | — | | — | | 300.0 | mg | 300.0 | mg |
| cellulose | | | | | | | | |
| Zinc nitrate hexahydrate | 22.3 | g | 22.3 | g | 22.3 | g | 22.3 | g |
| Methanol | 90.0 | g | 90.0 | g | 90.0 | g | 90.0 | g |
| Potassium hydroxide | 8.4 | g | 8.4 | g | 8.4 | g | 8.4 | g |
| Water | 60.0 | g | 60.0 | g | 60.0 | g | 60.0 | g |

### Methods for S, T, U and V

N—N diethyl benzamide (Method ‘T’) or N—N Diethyl Meta toluamide (Method ‘S’), HPC (only in method and ‘V’) and zinc nitrate were dissolved in methanol to form solution ‘A’. In yet another solution potassium hydroxide was dissolved in water to form solution ‘B’. Solution ‘A’ was added to solution ‘B’ at a controlled rate (0.2-0.5 ml/min) under continuous stirring at 500 rpm to form dispersion. The dispersion was further lyophilized to obtain fine powder. The concentrations of components of compositions ‘S’, ‘T’, ‘U’, ‘V’ are described in table 7.

### Example 6

Two tape stripping studies were conducted in order to know the dermatokinetics of different topical formulations of terbinafine of the present invention in wistar rats & guinea pigs, the results of the studies are described below:

### Study I

Wistar rats (female, 200-250 Gms) were used in the experiment for topical application of 30 mg the composition of ‘Method D’ containing 1.95 mg of Terbinafine. A commercially available terbinafine cream formulation was used as the reference for the study (B. No. 73002 T); reference dose of 195 mg contained 1.95 mg of terbinafine. The study was done for duration of 24 hours wherein the readings were observed at an interval of 0, 3, 6, 14 and 24 hrs. (n=6) rats were used, 6 rats/time point. The results are tabulated in table 8.

TABLE 8
Total concentration (μg) of terbinafine in stratum corneum with
treatment of reference and test formulation at various time points
in wistar rats (n = 5-6)

| | Total concentration of terbinafine (μg) (Mean ± SEM) | | | | |
|---|---|---|---|---|---|
| Formulation | 0 h | 3 h | 6 h | 14 h | 24 h |
| Reference | 0 ± 0 | 8.28 ± 1.82 | 8.86 ± 1.004 | 1.84 ± 0.42 | 1.5 ± 0.68 |
| Test | 0 ± 0 | 36.69 ± 6.20 | 38.55 ± 6.20 | 7.12 ± 1.71 | 7.90 ± 1.004 |

As seen in the table; test composition was retained at least 5 times higher than the reference composition in the stratum corneum at all the time points measured.

### Study II

Guinea pigs (either sex, 250-350 Gms) were used in the experiment for topical application of 30 mg the composition of ‘Method D’ containing 1.95 mg of Terbinafine. A commercially available cream formulation was used as reference for the study (B. No. 73002 T); reference dose of 195 mg contained 1.95 mg of Terbinafine. The study was done for duration of 24 hours wherein the readings were observed at an interval of 6 and 24 hrs. In total 35 (n=5) Guinea pigs were used. The results are tabulated in table 9.

TABLE 9
Total concentration (μg) of terbinafine in stratum corneum with
treatment of reference and test formulation at various time points
in guinea pigs (n = 5).

Total concentration
of terbinafine (μg)
(Mean ± SEM)

| Formulation | 6 h | 24 h |
|---|---|---|
| Reference | 14.77 ± 1.58 | 8.89 ± 1.36 |
| Test | 88.39 ± 12.59 | 30.98 ± 6.93 |

As seen in the table; test composition was retained at least 5 times and at least 3 times higher than the reference composition measured at time points of 6 h and 24 h respectively.

1. A composition having particles comprising:
a) inorganic element(s);
b) at least one or more active ingredient(s); and
c) optionally release rate modulating agent(s).

2. The composition according to claim 1, wherein the particles are nanoparticles or microparticles or mixtures thereof.

3. The composition according to claim 1, wherein the inorganic element is selected from group comprising of silica, alkaline metals, alkaline earth metals, transition metals, especially zinc, calcium, magnesium, titanium, silver, aluminium, and lanthanides, their salts, hydrates, as well as combinations thereof.

4. The composition according to claim 3, wherein the inorganic element is in the form of alkoxide, oxide, acetate, oxalate, ureate, or nitrate.

5. The composition according to claim 4, wherein the inorganic element is selected from a group comprising of zinc oxide, calcium carbonate, calcium oxide, calcium hydroxide, calcium bicarbonate or combinations thereof.

6. The composition according to claim 1, wherein release rate modulating agent(s) is selected from group of natural polymers, synthetic polymers, semi synthetic polymers, lipids, waxes and natural or synthetic gums, polysaccharides, monosaccharide, sugars, salts, proteins, peptides, polypeptides and combinations thereof.

7. The composition according to claim 6, wherein the release rate modulating agent is a natural, synthetic or semi-synthetic polymer especially biodegradable polymer or copolymer selected from the group of polyacrylate polymers, polyethylene oxide polymers, cellulose polymers, polyorthoesters, chitosan, polylactides, vinyl polymers and copolymers, alkylene oxide homopolymers polydioxanones, polyanhydrides, polycarbonates, polyesteramides, polyamides polyphosphazines, shellac derivatives and combinations thereof.

8. The composition according to claim 6, wherein the release rate modulating agent is a protein selected from gelatin, bovine serum albumin, human serum albumin and combinations thereof.

9. The composition according to claim 1, further comprising hydrophilic solvents, lipophilic solvents, humectants/plasticizers, thickening polymers, surfactants/emulsifiers, fragrances, preservatives, chelating agents, UV absorbers/filters, antioxidants, keratolytic agents, dihydroxyacetone or penetration enhancers, dispersing agents, deagglomerating agents and mixtures thereof.

10. The composition according to claim 2, wherein the particles have an average particle diameter of less than about 100 μm.

11. The composition of claim 2, wherein the particles are nanoparticles having an average particle diameter ranging from the group consisting of from about 1 nm to about 2000 nm, about 10 nm to about 200 nm, about 15 nm to about 150 nm.

12. The composition, according to claim 1, wherein the particles are synthesized by methods such as homogenization including high pressure homogenization, milling including ball milling, high shear wet milling, media milling, precipitation including supercritical fluid process, emulsification diffusion process, sol gel process, chemical or mechanical methods, aerosol flow reactor and the like thereof.

13. The composition according to claim 1, wherein the particles are synthesized by sol-gel process.

14. The composition according to claim 1, useful for topical application to the skin, mucosal surface like rectal, vaginal, surface of the eye, nasal passages, mouth or lips area or external ear.

15. The composition according to claim 14, wherein the composition is applied in a dosing frequency selected from the group consisting of two times a day, once a day, once in two days, thrice a week, twice a week and once in a week.

16. The composition of claim 1, wherein the composition has ability to be retained in the layers of skin especially in the stratum corneum, epidermis or dermis and combinations thereof.

17. The composition of claim 1, having controlled release profile selected from the group of monophasic, biphasic or multiphasic release profile.

18. The composition of claim 1, wherein, not more than 60% of the total amount of active ingredient is released within 2 hours and not less than 75% of the active ingredient is released within 14 hours when the composition is subjected to in-vitro dissolution studies.

19. A method of making composition according to claim 1 comprising:
a) dissolving active ingredient/s in a solvent to form solution (a);
b) dissolving inorganic metal salt in a solvent to form solution (b);
c) dissolving a release rate modulating agent in solvent to form solution (c); wherein an alkali hydroxide solution is included in any of the step of a), b) or c);
d) mixing solutions (a), (b) and (c) to form a precipitate; and
e) drying the precipitate formed in step (d) to form a dry powder composition.

20. A method of making composition of to claim 1, comprising the steps of:
a) dissolving inorganic metal salt in a solvent;
b) dissolving alkali hydroxide in a solvent;
c) dissolving active ingredient and polymer in a solvent;
d) adding alkali hydroxide of step (b) to solution of step (c);
e) adding inorganic metal salt of step (a) to prepared solution of step (d);
f) stirring the resultant solution of (e);
g) harvesting coarse aggregates by centrifugation and washing with water at least one time;
h) dispersing the nanoparticles in a mixture of solvent; and
i) thickening the nanoparticle dispersion obtained in (h) to form a gel.

21. The method according to claim 19, wherein the alkali hydroxide is selected from KOH, NaOH, LiOH, NH₄OH, Mg(OH)₂, hydrates thereof and combinations thereof.

22. The method according to claim 19, wherein drying is done by lyophilization, spray drying or spray freeze drying method or combinations thereof.

23. The method according to claim 19, wherein the solvent is selected from a group consisting of water, C₁-C₆ alcohol, methanol, ethanol, n-propanol, isopropanol, acetone, methylethyl ketone, tetrahydrofuran, benzene, toluene, o-xylene, m-xylene, p-xylene, mesitylene, diethyl ether, dichloromethane, chloroform, propylene glycol, triethanolamine and combinations thereof.

24. The composition according to claim 1, wherein the composition is a cream, lotion, gel, paste, powder, spray, foam, roll-on, oil, patch, suspension, ointment, deodorant or an aerosol.

25. The composition according to claim 24, wherein the composition is a dry powder for topical or mucosal applications.

26. The composition according to claim 25, wherein the composition is non-irritating and not visible when applied to skin or mucosal surfaces.

27. The composition according to claim 25, whereby the composition is non-gritty and easy to apply.

28. The composition according to claim 1, wherein the active ingredient is selected from antibiotics, antiviral agents, anti-fungals, analgesics, anorexics, antipsoriatics and acne treatment agents, anti herpes agents, antihelminthics, antiarthritics, antiasthmatic agents, anticonvulsants, antidepressants, antidiabetic agents, antidiarrheals, antihistamines, antiinflammatory agents, antimigraine preparations, antinauseants, antiandrogens, antisyphilictic agents, antineoplastics, antiparkinsonism drugs, antipruritics, antipsychotics, antipyretics, antispasmodics, anticholinergics, sympathomimetics, xanthine derivatives, cardiovascular preparations including potassium and calcium channel blockers, beta-blockers, alpha-blockers, and antiarrhythmics, antihypertensives, diuretics and antidiuretics, vasodilators including general coronary, peripheral and cerebral, central nervous system stimulants, vasoconstrictors, cough and cold preparations, including decongestants, hormones such as testosterone, estradiol and other steroids, including corticosteroids, hypnotics, immunosuppressives, muscle relaxants, parasympatholytics, psychostimulants, dermatitis herpetoformis suppressants, topical protectants, mosquito repellants, anti-lice agents, sedatives, tranquilizers, macromolecules such as proteins, polypeptides, polysaccharides, vaccines, antigens, antibodies and combinations thereof.

29. The composition according to claim 1, wherein the active ingredient is a cosmetic agent, selected from the group of antiageing agents, sunblocking agents, antiwrinkle agents, moisturizing agents, anti-dandruff agents especially selenium sulfide, vitamins, saccharides, oligosaccharides, hydrolysed or non-hydrolysed, modified or unmodified polysaccharides, amino acids, oligopeptides, peptides, hydrolysed or non-hydrolysed, polyamino acids, enzymes, branched or unbranched fatty acids and fatty alcohols, animal, plant or mineral waxes, ceramides and pseudoceramides, hydroxylated organic acids, antioxidants and free-radical scavengers, chelating agents, seborrhoea regulators, calmants, cationic surfactants, cationic polymers, amphoteric polymers, organomodified silicones, mineral, plant or animal oils, polyisobutenes and poly(.alpha.-olefins), fatty esters, anionic polymers in dissolved or dispersed form, nonionic polymers in dissolved or dispersed form, reducing agents, hair dyes or pigments, antioxidants, free radical scavengers, melanoregulators, tanning accelerators, depigmenting agents, skin-coloring agents, liporegulators, thinning agents, antiseborrhoeic agents, anti-UV agents, keratolytic agents, refreshing agents, cicatrizing agents, vascular protectors, antiperspirants, deodorants, skin conditioners, immunomodulators, nutrients and essential oils and perfumes, substance having a hair-care activity, agents for combating hair loss, hair dyes, hair bleaches, reducing agents for permanent waves, hair conditioners, nutrients or combinations thereof.

30. The composition according to claim 1, wherein the active ingredient is a peptide having molecular weight less than 100 kilo daltons and is selected from hair growth promoting actin binding peptides, RNA III Inhibiting peptides, cosmetically active peptides or peptide based colorants.

31. The composition according to claim 28, wherein the active ingredient is selected from antiviral agents, antifungal agents, antibacterial agents, immunosuppressants, antipsoriatics agents, antialopecia agents or antiacne agents.

32. The composition according to claim 31, wherein the antiviral agent is selected from group of acyclovir, ganciclovir, famciclovir, foscamet, inosine-(dimepranol-4-acetamidobenzoate), valganciclovir, valacyclovir, cidofovir, brivudin, antiretroviral active ingredients (nucleoside analog reverse-transcriptase inhibitors and derivatives) such as lamivudine, zalcitabine, didanosine, zidovudin, tenofovir, stavudin, abacavir, non-nucleoside analog reverse-transcriptase inhibitors such as amprenavir, indinavir, saquinavir, lopinavir, ritonavir, nelfinavir, amantadine, ribavirin, zanamivir, oseltamivir as well as any combinations thereof.

33. The composition according to claim 31, wherein antifungal agent is selected from allylamines (ammolfine, butenafine, naftifine, terbinafine), azoles (ketoconazole, fluconazole, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole), polyenes (nystatin, pimaricin, amphotericin B), pyrimidines (flucytosine), tetraenes (natamycin), thiocarbamates (tolnaftate), sulfonamides (mafenide, dapsone), glucan synthesis inhibitors (caspofungin), benzoic acid compounds, complexes and derivatives thereof (actofunicone) and other systemic or mucosal (griseofulvin, potassium iodide, Gentian Violet) and topical drugs (ciclopirox, ciclopirox olamine, haloprogin, undecylenate, silver sulfadiazine, undecylenic acid, undecylenic alkanolamide, Carbol-Fuchsin) as well as any combinations thereof.

34. The composition according to claim 31, wherein antibacterial agent includes, aclacinomycin, actinomycin, anthramycin, azaserine, azithromycin, bleomycin, cuctinomycin, carubicin, carzinophilin, chromomycines, clindamycin, ductinomycin, daunorubicin, 6-diazo-5-oxn-1-norieucin, doxorubicin, epirubicin, mitomycins, mycophenolsaure, mogalumycin, olivomycin, peplomycin, plicamycin, porfiromycin, puromycin, streptonigrin, streptozocin, tubercidin, ubenimex, zinostatin, zorubicin, aminoglycosides, polyenes, macrolid-antibiotics derivatives and combinations thereof.

35. The composition according to claim 31, wherein the antialopecia agent is selected from the group of minoxidil, cioteronel, diphencyprone and finasteride and combinations thereof.

36. The composition according to claim 31, wherein the antiacne agent is selected from the group of retinoids such as tretionin, isotretionin, adapalene, algestone, acetophenide, azelaic acid, benzoyl peroxide, cioteronel, cyproterone, motrtinide, resorcinol, tazarotene, tioxolone as well as any combinations thereof.

37. The composition according to claim 31, wherein the active ingredient is antipsoriatics agents selected from the group of dithranol, acitretin, ammonium salicylate, anthralin, 6-azauridine, bergapten, calcipotriene, chrysarobin, etritrenate, lonapalene, maxacalcitol, pyrogallol, tacalcitol and tazarotene as well as any combinations thereof.

38. The composition according to claim 31, wherein the active agent is an immunosuppressant selected from the group of tacrolimus, cyclosporine, sirolimus, alemtuzumab, azathioprine, basiliximab, brequinar, daclizumab, gusperimus, 6-mercaptopurine, mizoribine, muromonab CD3, pimecrolimus, rapamycin and combinations thereof.

39. The composition according to claim 1, wherein the active ingredient is a synthetic mosquito repellent selected from but not limited to N,N-diethyl-meta-toluamide (DEET), NN Diethyl Benzamide, 2,5-dimethyl-2,5-hexanediolbenzil, benzyl benzoate, 2,3,4,5-bis(butyl-2-ene)tetrahydrofurfural (MGK Repellent 11), butoxypolypropylene glycol, N-butylacetanilide, normal-butyl-6,6-dimethyl-5,6-dihydro-1,4-pyrone-2-carboxylate (Indalone), dibutyl adipate, dibutyl phthalate, di-normal-butyl succinate (Tabatrex), dimethyl carbate (endo,endo)-dimethyl bicyclo[2.2.1]hept-5-ene-2,3-dicarboxylate), dimethyl phthalate, 2-ethyl-2-butyl-1,3-propanediol, 2-ethyl-1,3-hexanediol (Rutgers 612), di-normal-propyl isocinchomeronate (MGK Repellent 326), 2-phenylcyclohexanol, p-methane-3,8-diol, and normal-propyl N,N-diethylsuccinamate and derivatives or combinations thereof or natural insect repellents selected from group of Dihydronepetalactone, Eucalyptus-derived p-menthan-3,8-diol (PMD) repellent, E-9-octadecenoic acid-derived compounds, extracts from limonene, citronella, eugenol, (+) eucamalol (1), (−)-1-epi-eucamalol, crude extract from plants such as eucalyptus maculata, vitex rotundifolia, cymbopogan, maltitol compound, peppermint oil, cinnamon oil, and nepetalaclone oil, Azadirachitin or other neem derived compounds and combinations thereof.

40. A composition according to claim 1, having particles comprising:
a) Inorganic element(s) in about 0.1% w/w to about
b) One or more active ingredient(s) in about 0.01% w/w to about 99.9% w/w.
c) Optionally release rate modulating agent(s) in about 0.001% w/w to about 75% w/w of the total weight.

41. A composition having particles comprising:
a) inorganic element(s);
b) at least one active ingredient selected from acyclovir, terbinafine, clindamycin, N—N-diethyl-meta-toluamide (DEET), N—N-Diethyl Benzamide or actin binding peptide homologous to Tβ4 or analogs thereof and
c) optionally release rate modulating agent(s).

42. Dry powder composition comprising active ingredient selected from the group of acyclovir, terbinafine, clindamycin, N,N-diethyl-meta-toluamide (DEET) or N,N-Diethyl Benzamide for topical or mucosal application.

43. Dry powder composition according to claim 42, further comprising inorganic element and optionally release rate modulating agent(s).

44. Dry powder composition according to claim 43, formulated as nanoparticles or microparticles or mixtures thereof.

45. Dry powder composition according to claim 42, wherein active ingredient is present in the dosage range from about 1% w/w to about 95% w/w of the total weight.

46. Dry powder composition according to claim 42, wherein the composition is applied in the dosing frequency selected from the group consisting of two times a day, once a day, once in two days, thrice a week, twice a week and once in a week.

47. Dry powder composition according to claim 42, wherein the composition has ability to retain in the upper layers of skin.

48. Dry powder composition according to claim 42, having controlled release, especially monophasic, biphasic, multiphasic release profiles.

49. Dry powder composition comprising actin binding peptide suitable for promotion of hair growth in humans especially a homologous Tβ4 peptide or analogs thereof for topical or mucosal application.

50. Dry powder composition according to claim 49, further comprising inorganic element and optionally release rate modulating agent(s).

51. Dry powder composition according to claim 50, formulated as nanoparticles or microparticles or mixtures thereof.

52. Dry powder composition according to claim 49, wherein the required dosage of actin binding peptide, homologous to Tβ4 or analogs thereof is between 0.001% w/w to about 20% w/w of the total weight.

53. Dry powder composition according to claim 49, wherein the composition is applied in the dosing frequency selected from the group consisting of two times a day, once a day, once in two days, thrice a week, twice a week and once in a week.

54. Dry powder composition according to claim 49, wherein the composition has ability to retain in the upper layers of skin.

55. Dry powder composition according to claim 49, having controlled release profile selected from the group of monophasic, biphasic, multiphasic release profiles.

56. A kit comprising a delivery device; the composition of claim 1; and instructions for its use; for the delivery of the composition to topical or mucosal surfaces.

57. A kit according to claim 56, wherein the delivery device comprises a pressurized or non-pressurized dispensing device or applicator or mechanical device, which delivers the composition to the topical or mucosal surfaces.

58. A kit according to claim 57, wherein the delivery device comprises a pressurized or non-pressurized dispensing device which delivers metered dose of the composition to the topical or mucosal surfaces.

59. The composition according to claim 1, wherein the composition further includes occlusive patches for the prevention of particles escaping to the exterior of the application area.

60. Particles comprising:
a) inorganic element(s)
b) one or more active ingredient(s) and
c) optionally release rate modulating agent(s).

61. The method according to claim 20, wherein the alkali hydroxide is selected from KOH, NaOH, LiOH, NH₄OH, Mg(OH)₂, hydrates thereof and combinations thereof.

62. The method according to claim 20, wherein the solvent is selected from a group consisting of water, C₁-C₆ alcohol, methanol, ethanol, n-propanol, isopropanol, acetone, methylethyl ketone, tetrahydrofuran, benzene, toluene, o-xylene, m-xylene, p-xylene, mesitylene, diethyl ether, dichloromethane, chloroform, propylene glycol, triethanolamine and combinations thereof.
